(19) United States
(12) Patent Application Publication
(10) Pub. No.: US 2009/0011117 A1
(43) Pub. Date: Jan. 8, 2009

(54) METHODS FOR TEXTURING A SURFACE OF AN ENDOVASCULAR IMPLANT

(75) Inventors: Anthony I. Nunez, Beachwood, OH (US); Harry D. Rowland, East Peoria, IL (US)

Correspondence Address: Patent Docket Department;Armstrong Teasdale LLP, One Metropolitan Square, Suite 2600, St. Louis, MO, 63102-2740, US

(73) Assignee: ENDOTRONIX, INC., Peoria, IL (US)

(21) Appl. No.: 12/167,061

(22) Filed: Jul. 2, 2008

Related U.S. Application Data
(60) Provisional application No. 60/947,909, filed on Jul. 3, 2007.

(51) Int. Cl. A61F 2/06

(52) U.S. Cl. 427/2.31; 264/271.1

(57) ABSTRACT

A method for fabricating an endovascular implant includes providing a mold having a controlled topography. A wire is positioned about at least a portion of the mold. A polymeric material coating is applied to cover at least a portion of the mold. The wire and the polymeric material coating are released from the mold, wherein the polymeric material coating has a controlled topography at least partially replicating the controlled topography of the mold.

### CROSS REFERENCE TO RELATED APPLICATIONS

This application claims the benefit of U.S. Provisional Application No. 60/947,909, filed Jul. 3, 2007, which is hereby incorporated by reference in its entirety.

### BACKGROUND OF THE INVENTION

The subject matter disclosed herein relates generally to a system and a method for creating a controlled topography on an arbitrary surface and, more particularly, to a system and a method for texturing a surface of an endovascular implant, such as a stent or graft.

Implantable tubular structures, such as endovascular stents or grafts, often develop excessive neointimal growth at the anastomotic interface and thus experience early failure. At least one known tubular structure includes deactivated heparin bonded to an inner surface of the tubular structure to reduce a tendency toward neointimal growth. Alternative or additional methods for preventing or limiting such neointimal growth are desired.

### BRIEF DESCRIPTION OF THE INVENTION

In one aspect, a method for fabricating an endovascular implant is provided. The method includes providing a mold having a controlled topography. A wire is positioned about at least a portion of the mold. A polymeric material coating is applied to cover at least a portion of the mold. The wire and the polymeric material coating are released from the mold such that the polymeric material coating has a controlled topography at least partially replicating the controlled topography of the mold.

In another aspect, a method is provided for fabricating an endovascular implant having a controlled topography. The method includes providing a structure having an outer surface with a controlled topography. A wire is wrapped about at least a portion of the outer surface of the structure. A polymeric material coating having a controlled topography is applied to the structure. The wire and the polymeric material coating are released from the structure.

In yet another aspect, a method for fabricating a three-dimensional mold structure having an outer surface with a controlled topography is provided. The method includes forming a first layer of polymeric material. A second layer of polymeric material is applied on the first layer of polymeric material. A mask structure is formed on the second layer. The mask structure is attached to a third layer of material such that the second layer contacts the third layer. The first layer of polymeric material is removed such that the mask structure defines a plurality of voids providing access to the third layer and a material is deposited within the plurality of voids defined by the mask structure.

In another aspect, a method is provided for fabricating an endovascular graft having a surface with a controlled topography. The method includes providing a mold structure. A polymeric material coating having a controlled topography is applied to the mold structure to cover at least a portion of the mold structure. The polymeric material coating is released from the mold structure.

### BRIEF DESCRIPTION OF THE DRAWINGS

FIGS. 1-8 are schematic sectional views illustrating an exemplary system and method for forming a controlled topography on an arbitrary surface using a mask structure;

FIG. 9 is a schematic perspective view of a cylindrical mold structure;

FIG. 10 is a schematic perspective view of a portion of a smooth outer surface of the cylindrical mold structure shown in FIG. 9;

FIG. 11 is a schematic perspective view of a cylindrical mold structure having a controlled surface topography;

FIG. 12 is a schematic perspective view of a portion of a textured outer surface of the cylindrical mold structure shown in FIG. 11;

FIG. 13 is a schematic perspective view of a polymeric material applied to the cylindrical mold structure shown in FIG. 11;

FIG. 14 is a schematic perspective view of an endovascular implant having a controlled surface topography formed from the polymeric material applied to the cylindrical mold structure as shown in FIG. 13; and

FIG. 15 is a schematic perspective view of a portion of a textured inner surface of the endovascular implant shown in FIG. 14.

### DETAILED DESCRIPTION OF THE INVENTION

The present disclosure describes a system and a method for controlling a surface topography of an endovascular implant, such as a stent or a graft, which facilitates controlling properties of blood flow at or near a surface of the endovascular implant. Patterning and/or texturing one or more surfaces of the endovascular implant may facilitate modifying coagulation properties of the endovascular implant to prevent or limit endothelialization and/or reduce a risk of thrombosis and/or embolism. Further, patterning and/or texturing one or more surfaces of the endovascular implant may also facilitate modifying flow properties of blood at or near the one or more surfaces to promote or reduce slip at or near the surfaces and desirably alter a laminar flow characteristic and/or a turbulent flow characteristic of the blood through the endovascular implant.

The subject matter described herein is in reference to forming a controlled surface topography on an endovascular implant, such as a stent or graft. However, it should be apparent to those skilled in the art and guided by the teachings herein provided that the subject matter described herein may likewise be applicable to other suitable implantable devices including, without limitation, heart valves, sensors, and closure devices.

As used herein, references to “controlled topography” and “controlled surface topography” are to be understood to refer to a formation of a patterned and/or textured surface on an arbitrary object, such as an endovascular implant, using a system and/or a method that allows feature dimensions to be controlled to produce a patterned and/or textured surface having undulations, regions, ridges and/or valleys, for example, having desired dimensions including a height, a width, and/or a length.

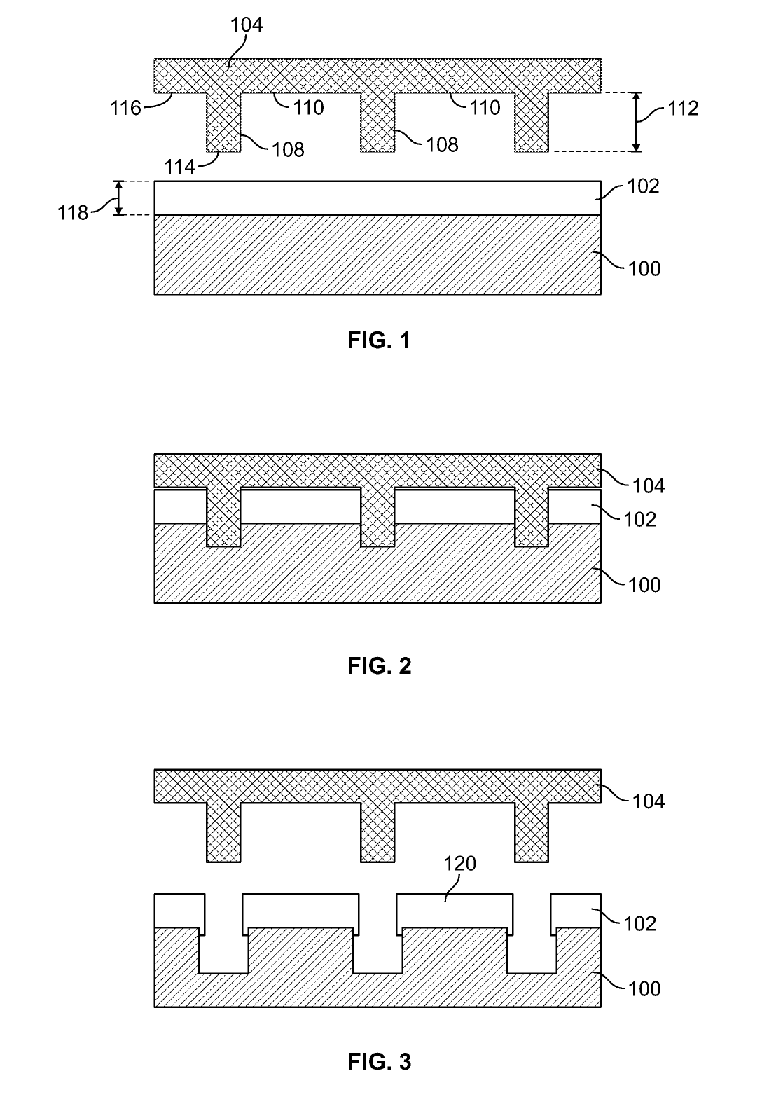

FIGS. 1-8 are schematic sectional views illustrating an exemplary system and method for forming a controlled topography on an arbitrary surface using a mask structure, herein described using an embossing or molding process. Alternative embodiments may pattern the mask structure with processes other than embossing or molding, such as laser ablation, or lithography and etch processes. FIG. 1 shows a first layer 100 of deformable material and a second layer 102 of deformable material applied to or formed on first layer 100. A stamp structure 104 is configured to mold at least second layer 102. In a particular embodiment, stamp structure 104 is configured to mold both second layer 102 and first layer 100. Stamp structure 104 has a desired or selected three-dimensional surface topography with one or more raised regions 108 and one or more recessed regions 110. In one embodiment, a distance 112 between a surface 114 of raised region 108 and a surface 116 of corresponding recessed region 110 is greater than a thickness 118 of second layer 102. Stamp structure 104 is made of a suitable material, such as a metal, polymer, silicon, or ceramic material, or a combination of suitable materials. In one embodiment, second layer 102 and/or stamp structure 104 is coated with an interface layer (not shown) to reduce adhesion of second layer 102 to stamp structure 104 during the molding process. In a particular embodiment, second layer 102 is coated with an interface layer (not shown) to create a different chemical functionality or hydrophilicity/hydrophobicity than a respective chemical functionality or hydrophilicity/hydrophobicity of first layer 100.

FIG. 2 shows stamp structure 104 controllably embossing or molding second layer 102 and first layer 100. In one embodiment, second layer 102 and first layer 100 are molded in the same process. In alternative embodiments, second layer 102 is molded before second layer 102 is applied to first layer 100. After second layer 102 is molded, first layer 100 is molded. In a particular embodiment, second layer 102 has a lower modulus or softening temperature than a modulus or softening temperature of first layer 100. Prior to removing stamp structure 104, first layer 100 and/or second layer 102 is solidified by a suitable process, such as a cooling, solvent evaporation, or thermal radiation or electromagnetic radiation process. In this embodiment, the molding process forms a discontinuous second layer 102, as shown in FIG. 3. Referring further to FIG. 3, stamp structure 104 is removed from first layer 100 and second layer 102 after the molding process is complete and first layer 100 and/or second layer 102 has solidified. An embossing process known to those skilled in the art and guided by the teachings herein provided forms a patterned mask structure 120 from second layer 102.

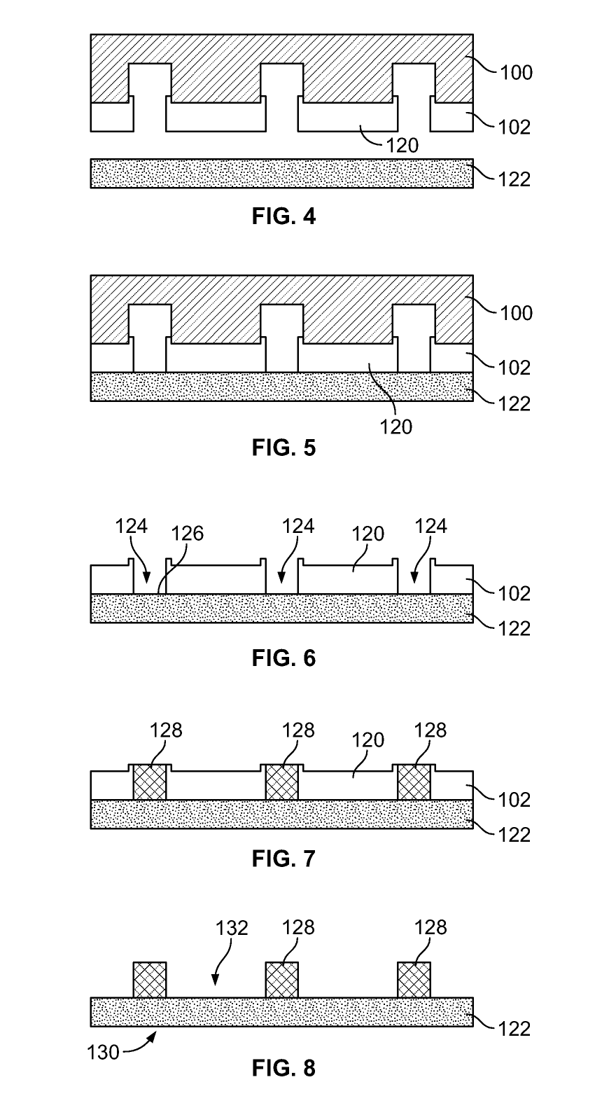

As shown in FIG. 4, mask structure 120 is attached or coupled to a third layer 122 of material. Third layer 122 is formed of a suitable material, such as a metal, polymer, silicon, or ceramic material, or a combination of suitable materials. In one embodiment, third layer 122 is coated with a thin layer of metal (not shown) to act as a seed layer for electroplating. In one embodiment, mask structure 120 is coated with an adhesive layer (not shown) prior to contact with third layer 122. Mask structure 120 is attached to third layer 122, as shown in FIG. 5. With mask structure 120 attached to third layer 122, first layer 100 is removed from second layer 102, forming voids 124 that provide access to a first surface 126 of third layer 122 through mask structure 120. In one embodiment, first layer 100 is selectively removed using a suitable chemical wet etch process known to those skilled in the art and guided by the teachings herein provided, without modifying second layer 102 or third layer 122. In one embodiment, mask structure 120 remains as a controlled topography on the arbitrary surface. In a particular embodiment, mask structure 120 is post-processed chemically, thermally, and/or using ultraviolet radiation to change a functionality of mask structure 120.

Referring further to FIG. 7, a suitable material 128 is deposited in voids 124 (shown in FIG. 6) formed in mask structure 120 and attaches to third layer 122. In one embodiment, material 128 is deposited using a suitable electroplating process known to those skilled in the art and guided by the teachings herein provided. FIG. 8 shows mask structure 120 removed from third layer 122. In one embodiment, mask structure 120 is removed from third layer 122 using a suitable process, such as a chemical wet etch process, that does not remove or modify third layer 122 or material 128 attached to third layer 122. The resulting structure 130 defines an arbitrary surface 132 having a controlled topography, as shown in FIG. 8. In one embodiment, third layer 122 and material 128 include the same metal material such that arbitrary surface 132 with the controlled topography provides a metal mold useful for embossing or molding curved surfaces and/or planar surfaces.

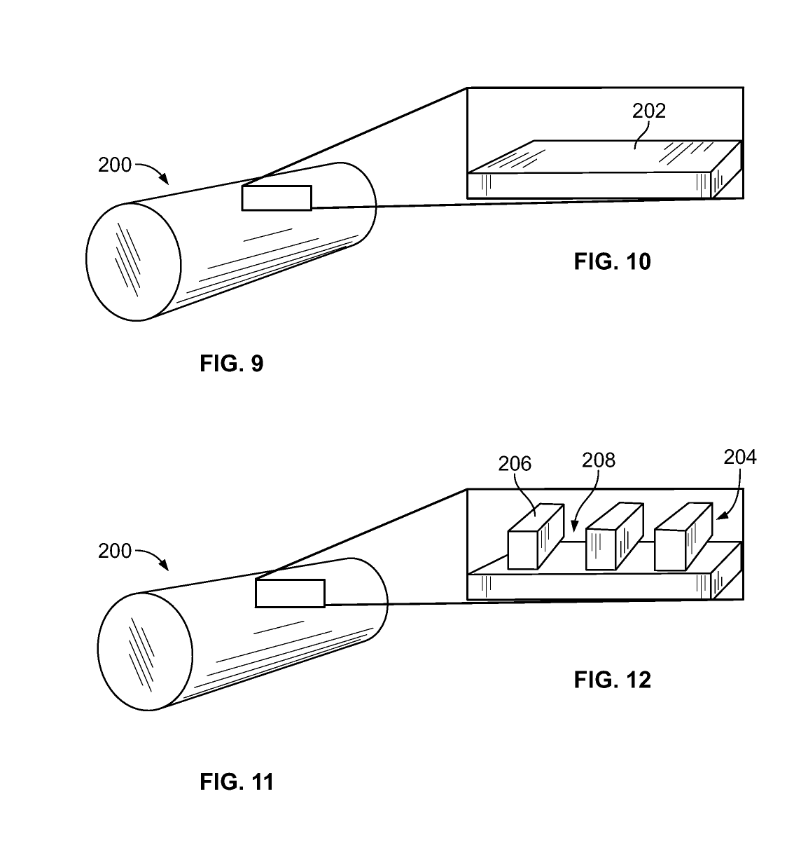

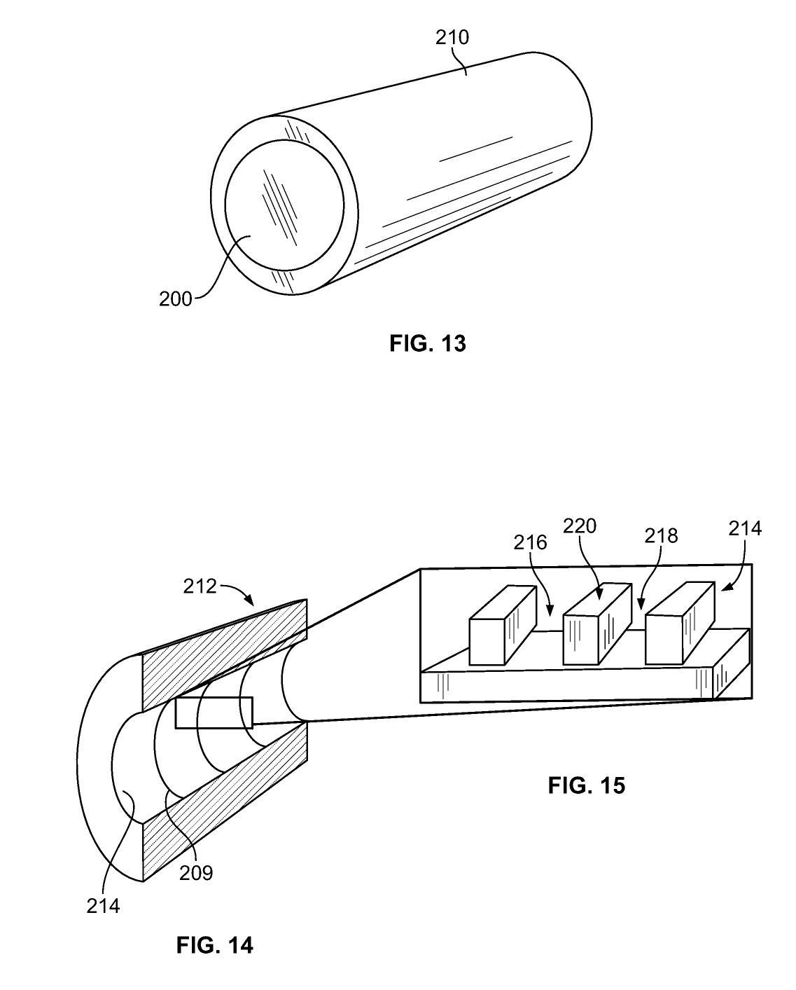

FIG. 9 is a schematic perspective view of a cylindrical mold structure 200. FIG. 10 is a schematic perspective view of a portion of a smooth or unpatterned outer surface 202 of cylindrical mold structure 200 shown in FIG. 9. A controlled surface topography is formed on unpatterned outer surface 202 of mold structure 200, as described herein, such that mold structure 200 is suitable for forming or fabricating an endovascular implant, such as a stent or graft, having a patterned and/or textured inner surface. Although the mold or mold structure shown in FIGS. 9 and 10 is described herein as a cylindrical mold structure 200, it should be apparent to those skilled in the art and guided by the teachings herein provided that, in alternative embodiments, the mold or mold structure may have any suitable arbitrary shape including, without limitation, a suitable three-dimensional shape for forming or fabricating an object, including an implantable medical device, such as a heart valve, a pacemaker covering, or a septal occluder, for example, having one or more patterned and/or textured surfaces, as desired.

FIG. 11 is a schematic perspective view of a cylindrical mold structure having a controlled surface topography. FIG. 12 is a schematic perspective view of a portion of a patterned and/or textured outer surface of the cylindrical mold structure shown in FIG. 11. Referring to FIGS. 11 and 12, mold structure 200 is molded to form a patterned and/or textured outer surface 204 having a desired or selected three-dimensional surface topography with one or more raised regions 206 and one or more recessed regions 208.

In one embodiment, a suitable wire 209 (shown in FIG. 14) is positioned or wrapped about at least a portion of outer surface 204. A suitable polymeric material 210 is applied to, such as wrapped about, at least a portion of outer surface 204 of mold structure 200 to fabricate an endovascular implant 212, a portion of which is shown schematically in FIGS. 14 and 15, having a controlled surface topography including a textured inner surface. In this embodiment, wire 209 is coated with or covered by polymeric material 210 to cover mold structure 200 and create a covered wire. In alternative embodiments, polymeric material 210 is applied directly to mold structure without first positioning wire 209 about at least a portion of outer surface 204. Suitable polymeric materials include, without limitation, a low-porosity polymer, polytetrafluoroethylene, porous polytetrafluoroethylene, and/or expanded polytetrafluoroethylene, as well as other suitable polymers. In one embodiment, a plurality of polymeric material coatings are wrapped about at least a portion of mold structure 200. Polymeric material 210 may be heated and/or pressed onto mold structure 200 such that textured outer surface 204 is transferred to or replicated on an inner surface 214 of polymeric material 210 forming endovascular implant 212. As shown in FIG. 15, inner surface 214 of endovascular implant 212 has a controlled surface topography 216 formed from outer surface 204 of mold structure 200. More specifically, referring to FIG. 15, endovascular implant 212 is molded to form patterned and/or textured inner surface 214 having a desired or selected three-dimensional surface topography 216 with one or more recessed regions 218 formed or molded by a corresponding raised region 206 of mold structure 200 and one or more raised regions 220 formed or molded by a corresponding recessed region 208 of mold structure 200. In a further embodiment, endovascular implant 212 is formed with a textured outer surface using a suitably fabricated mold structure.

Endovascular implant 212 may be textured with a controlled topography including features having dimensions ranging from about 10 nanometers (nm) to about 100 microns (μm) such that properties of blood flow at or near inner surface 214 of endovascular implant 212 are modified. Patterning inner surface 214 facilitates modifying coagulation properties of endovascular implant 212 to prevent or limit endothelialization and/or reduce a risk of thrombosis and/or embolism. Patterning inner surface 214 may also facilitate modifying flow properties of blood at or near inner surface 214 to promote or reduce slip at or near inner surface 214 to alter a laminar flow characteristic and/or a turbulent flow characteristic of blood through endovascular implant 212. Controlled surface topography 216 may also form small wells that may be filled with a slow release polymer that has been impregnated with an antimetabolite substance that inhibits cell division, such as Tacrolimus or Sirolimus. The filled wells may then be covered with a porous polymer layer to allow a time-controlled release of a drug. In further embodiments, an external surface of a pressure sensor (not shown) coupled to endovascular implant 212 may be coated with a deactivated heparin bonded material to form an anti-coagulation or antimetabolite coating.

In alternative embodiments, a sheet of polymeric material 210 may be textured using mold structure 200 such that after molding, a controlled topography of mold structure 200 is transferred to one or more surfaces of the sheet of polymeric material 210. The textured sheet of polymeric material 210 may then be wrapped around mold structure 200 with unpatterned surface 202. The sheet of polymeric material 210 may be formed about mold structure 200 at temperatures and/or pressures that do not appreciably deform the controlled topography such that endovascular implant 212 may be formed having a controlled topography. In further alternative embodiments, an endovascular implant having one or more textured surfaces may be formed by wrapping one or more sheets of a textured polymeric material about at least a portion of a suitably fabricated mold structure.

In one embodiment, a method for fabricating an endovascular implant, such as a tubular intraluminal graft, with controlled topography includes providing a tubular cylindrical mold structure that has controlled topography, wrapping a wire about the tubular cylindrical mold structure, applying a polymeric coating to the tubular cylindrical mold structure, and releasing the wire and the polymeric material coating from the tubular cylindrical mold structure such that the polymeric material coating has a controlled topography formed by the controlled topography of the tubular cylindrical mold structure. In one embodiment, at least a portion of a length of the wire is coated with the polymeric material coating to create a covered wire. The polymeric material coating may include a low-porosity polymer, polytetrafluoroethylene, porous polytetrafluoroethylene, and/or expanded polytetrafluoroethylene, as well as other suitable polymers. In one embodiment, multiple polymeric material coatings are wrapped around the tubular cylindrical mold. One or more of the multiple polymeric material coatings may have a controlled topography on an interior surface and/or an exterior surface of the polymeric material coating.

In one embodiment, a method for fabricating an endovascular implant, such as a tubular intraluminal graft, with controlled topography includes providing a tubular cylindrical structure, wrapping a wire about the tubular cylindrical structure, applying a polymeric material coating having a controlled topography to cover the tubular cylindrical structure, and releasing the wire and the polymeric coating from the tubular cylindrical structure. The controlled topography of the polymeric material coating may be formed by a molding, embossing, solid forming, and/or nanoimprint lithography process known to those skilled in the art and guided by the teachings herein provided. In one embodiment, the controlled topography includes features having dimensions ranging from about 10 mm to about 100 μm. The controlled topography of the polymeric material coating may be formed by any suitable printing process known to those skilled in the art and guided by the teachings herein provided.

In one embodiment, a method for making a tubular mold structure with a controlled topography includes providing a first layer of polymeric material, applying a second layer of a material, such as a polymeric material, to the first layer, patterning at least the second layer such that the second layer is made discontinuous to form a mask structure, attaching the mask structure to a third layer such that the second layer contacts the third layer, removing the first layer such that the mask structure allows access to the third layer through a plurality of voids; depositing a material within the voids defined by the mask structure, and removing the second layer to provide the third layer having a controlled surface topography including the material. The first and second layers may be patterned by molding, embossing, and/or laser writing. Alternatively, the first and second layers are patterned by lithography or wet or dry chemical etching.

In one embodiment, the first or second layer materials are selectively removed without altering other layers. Further, the first and second layers may be patterned in multiple steps. In a particular embodiment, a thin metal layer is deposited on the third surface. The material is deposited in the voids using an electroplating process. Alternatively, the material is deposited using any suitable printing process including, without limitation, an ink jetting, spin-coating, casting, lithography, gravure printing, screen printing, roll coating, gap coating, rod coating, extrusion coating, dip coating, curtain coating, air knife coating, impact printing, stamping, roll-to-roll printing, and/or contact printing process. In one embodiment, the second layer mask structure is patterned with an adhesive agent using contact printing prior to being attached to the third layer. The controlled topography may be created on the external surface and/or the internal surface of the tubular mold structure. Further, the controlled topography may be created on a planar surface or a three-dimensional curved surface. In a particular embodiment, the second layer of material is not removed from the third layer but instead forms the controlled surface topography on the tubular mold structure.

In one embodiment, a method for fabricating an endovascular implant, such as a stent, a graft, a heart valve, or a closure device, with controlled topography includes providing a mold structure, applying a polymeric material coating having a controlled topography to cover the mold structure, and releasing the polymeric material coating from the mold structure. The controlled topography of the polymeric material coating is formed by a molding, embossing, solid forming, and/or nanoimprint lithography process. The controlled topography includes features having dimensions ranging from about 10 nm to about 100 μm. The controlled topography of the polymeric material coating is formed by any suitable printing, molding, embossing, solid forming, and/or nanoimprint lithography process known to those skilled in the art and guided by the teachings herein provided. In one embodiment, a wire is wrapped around the mold structure. Further, the wire may be coated with the polymeric material coating to create a covered wire. The polymeric material coating may include a low-porosity polymer, polytetrafluoroethylene, porous polytetrafluoroethylene, and/or expanded polytetrafluoroethylene, as well as other suitable polymeric materials. In one embodiment, multiple polymeric material coatings are wrapped around the mold structure. One or more of the multiple polymeric material coatings has a controlled topography on at least a portion of the interior surface and/or exterior surface. A heparin-bonded coating and/or a drug eluting coating may also be applied to one or more surfaces of the endovascular implant.

As described above, conventional implantable tubular structures or implants, such as endovascular stents and grafts, may develop excessive neointimal growth at an anastomotic interface and thus experience early failure. At least one conventional tubular structure may include deactivated heparin bonded to a surface of the tubular structure to reduce a tendency toward neointimal growth. However, a laminar flow of fluid, such as blood, created at an inner surface/blood flow interface by microscopically textured features, such as undulations, regions, ridges and/or valleys, may assist in reducing the neointimal growth. Further, smaller coronary stent surfaces may be modified to induce laminar surface flow thereby reducing the formation of neointima, which reduces a restenosis rate and aids in ensuring patency. Surface modifications may also include a special combination of surface wells for antimetabolite slow release polymers adjacent to or combined with textured ridges, thereby enhancing the field/edge effect of the drug polymer. Alternatively, certain aspects of the coronary stent surface can be modified to increase neointimal formation selectively so as not to have a bare stent surface due to a drug eluting effect. Such residual bare metal surface may promote undesired subacute thrombosis in conventional implants. Flat polymeric surfaces on an atrial wall or a ventricular septal wall may also be textured with a spiral format or pattern of ridges to create a washing effect of the blood as the blood is directed from a center of the flat polymeric surface toward an edge of the stent. The spiral ridges may be fabricated such that the spiral ridges spiral outwardly and have a higher profile at a center and a lower profile at the edge. Flat valvular surfaces may be textured to enhance laminar flow characteristics thereby decreasing stress and/or strain on an edge of the valvular surfaces as they coapt. Valvular failure may be reduced by reducing high pressure points and low pressure points at the edge. The venturi effect that increases the coronary blood flow can be enhanced by placing the microridges and/or the macroridges on an aspect and/or an outer aspect of the valve leaflets.

The methods for texturing a surface of a polymeric material, as described herein, provide a benefit of reducing a tendency towards developing excessive neointimal growth by changing one or more surface characteristics and/or redirecting turbulent flow toward laminar flow.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for fabricating an endovascular implant, the method comprising:
providing a mold having a controlled topography;
positioning a wire about at least a portion of the mold;
applying a polymeric material coating to cover at least a portion of the mold; and
releasing the wire and the polymeric material coating from the mold, the polymeric material coating having a controlled topography at least partially replicating the controlled topography of the mold.

2. A method in accordance with claim 1 wherein at least one of a low-porosity polymer, polytetrafluoroethylene, porous polytetrafluoroethylene, and expanded polytetrafluoroethylene is applied to cover at least a portion of the cylindrical mold.

3. A method in accordance with claim 1 wherein a plurality of polymeric coatings are applied to cover at least a portion of the cylindrical mold.

4. A method for fabricating an endovascular implant having a controlled topography, the method comprising:
providing a structure having an outer surface with a controlled topography;
wrapping a wire about at least a portion of the outer surface of the structure;
applying a polymeric material coating having a controlled topography to the structure; and
releasing the wire and the polymeric material coating from the structure.

5. A method in accordance with claim 4 wherein the controlled topography of the polymeric material coating is formed by one of a molding, embossing, solid forming, and nanoimprint lithography process.

6. A method in accordance with claim 4 further comprising form a plurality of features on an inner surface of the polymeric material coating, each feature of the plurality of features having a height of about 10 nanometers to about 100 microns.

7. A method in accordance with claim 4 wherein the controlled topography of the polymeric material coating is formed using a printing process.

8. A method in accordance with claim 4 wherein applying a polymeric material coating comprises coating a length of wire with the polymeric coating to create a covered wire.

9. A method in accordance with claim 4 wherein at least one of a low-porosity polymer, polytetrafluoroethylene, porous polytetrafluoroethylene, and expanded polytetrafluoroethylene is applied to the cylindrical mold.

10. A method in accordance with claim 4 wherein the controlled topography of the polymeric material coating is formed by one of a molding, embossing, solid forming, and nanoimprint lithography process.

11. A method in accordance with claim 4 wherein a plurality of polymeric material coatings are applied to the cylindrical structure.

12. A method in accordance with claim 11 wherein at least one polymeric material coating of the plurality of polymeric material coatings has a controlled topography on at least one of an interior surface and an exterior surface.

13. A method for fabricating a three-dimensional mold structure having an outer surface with a controlled topography, the method comprising:
forming a first layer of polymeric material;
applying a second layer of polymeric material on the first layer of polymeric material;
forming a mask structure on the second layer;
attaching the mask structure to a third layer of material such that the second layer contacts the third layer;
removing the first layer of polymeric material such that the mask structure defines a plurality of voids providing access to the third layer; and
depositing a material within the plurality of voids defined by the mask structure.

14. A method in accordance with claim 13 further comprising removing the second layer of polymeric material to provide the third layer having a controlled topography including the deposited material.

15. A method in accordance with claim 13 wherein forming a mask structure on the second layer comprises patterning the first layer and the second layer such that the second layer is made discontinuous.

16. A method in accordance with claim 13 wherein at least one of the first layer and the second layer are patterned using one of a molding, embossing, laser writing, lithography, wet chemical etching and dry chemical etching process.

17. A method in accordance with claim 13 wherein at least one of the first layer and the second layer are selectively removed.

18. A method in accordance with claim 13 further comprising depositing a thin metal layer on the third layer.

19. A method in accordance with claim 13 wherein the deposited material is deposited using an electroplating process.

20. A method in accordance with claim 13 wherein the deposited material is deposited using one of an ink jetting, spin-coating, casting, lithography, gravure printing, screen printing, roll coating, gap coating, rod coating, extrusion coating, dip coating, curtain coating, air knife coating, impact printing, stamping, roll-to-roll printing, and contact printing process.

21. A method in accordance with claim 13 further comprising patterning the mask structure with an adhesive agent using contact printing prior to attaching to the third layer.

22. A method in accordance with claim 13 wherein the controlled topography is created on at least one of an external surface and an internal surface of the tubular mold structure.

23. A method in accordance with claim 13 wherein the controlled topography is created on one of a planar surface and a curved surface of the tubular mold structure.

24. A method for fabricating an endovascular graft having a surface with a controlled topography, the method comprising:
providing a mold structure;
applying a polymeric material coating having a controlled topography to the mold structure to cover at least a portion of the mold structure; and
releasing the polymeric material coating from the mold structure.

25. A method in accordance with claim 24 wherein the controlled topography of the polymeric material coating is formed by one of a printing, molding, embossing, solid forming, and nanoimprint lithography process.

26. A method in accordance with claim 24 wherein a plurality of polymeric material coatings are applied to the mold structure, at least one polymeric material coating of the plurality of polymeric material coatings has a controlled topography on one of an interior surface and an exterior surface.
